(19) United States
(12) Patent Application Publication
(10) Pub. No.: US 2008/0009846 A1
(43) Pub. Date: Jan. 10, 2008

(54) Electrosurgical return electrode with an involuted edge

(75) Inventor: Arlen K. Ward, Thornton, CO (US)

Correspondence Address: COVIDIEN, 60 MIDDLETOWN AVENUE, NORTH HAVEN, CT, 06473, US

(73) Assignee: SHERWOOD SERVICES AG

(21) Appl. No.: 11/481,662

(22) Filed: Jul. 6, 2006

(51) Int. Cl. A61B 18/16

(52) U.S. Cl. 606/32

(57) ABSTRACT

An electrosurgical return electrode is disclosed. The electrosurgical return electrode includes a conductive pad. The conductive pad includes a perimeter which has at least one involuted peripheral edge which is configured to reduce the current density of the conductive pad at the perimeter of the conductive pad. The involuted peripheral edge includes a depth and a width. The depth of the involuted edge is at least about 30% of the width of the involuted edge.

### BACKGROUND

The present disclosure is directed to electrosurgical apparatus, methods and systems, and, in particular, to an electrosurgical return electrode including an involuted edge.

During electrosurgery, a source or active electrode delivers energy, such as radio frequency energy, from an electrosurgical generator to the patient and a return electrode carries the current back to the electrosurgical generator. In monopolar electrosurgery, the source electrode is typically a hand-held instrument placed by the surgeon at the surgical site and the high current density flow at this electrode creates the desired surgical effect of cutting and/or coagulating tissue. The patient return electrode is placed at a remote site from the source electrode and may be in the form of a pad adhesively adhered to the patient.

The return electrode typically has a large patient contact surface area to minimize heating at that site since the larger the surface area, the lower the current density and the lower the intensity of the heat. The size of return electrodes are based on assumptions of the maximum current seen in surgery and the duty cycle (e.g., the percentage of time the generator is on) during the procedure. The first types of return electrodes were in the form of large metal plates covered with conductive jelly. Later, adhesive electrodes were developed with a single metal foil covered with conductive jelly or conductive adhesive. However, one issue with these adhesive electrodes was that if a portion peeled from the patient, the contact area of the electrode with the patient decreased, thereby increasing the current density at the adhered portion and, in turn, increasing the heat at the electrode site. This increased the risk of a patient burn under the adhered portion of the return electrode if the tissue was heated beyond the point where circulation of blood could cool the skin.

To address this problem, split return electrodes and hardware circuits, generically called Return Electrode Contact Quality Monitors (RECQMs), were developed. These split electrodes typically consist of two separate conductive foils arranged as two halves of a single return electrode. The hardware circuit uses an AC signal between the two electrode halves to measure the impedance therebetween. This impedance measurement is indicative of how well the return electrode is adhered to the patient since the impedance between the two halves is directly related to the area of patient contact. That is, if the electrode begins to peel from the patient, the impedance increases since the contact area of the electrode decreases. Current RECQMs are designed to sense this change in impedance so that when the percentage increase in impedance exceeds a predetermined value or the measured impedance exceeds a threshold level, the electrosurgical generator is shut down and/or an alarm is sounded to reduce the chances of burning the patient.

### SUMMARY

The present disclosure relates to an electrosurgical return electrode. The electrosurgical return electrode includes a conductive pad. The conductive pad includes a perimeter having at least one involuted peripheral edge. The involuted peripheral edge is configured to reduce the current density of the conductive pad at the perimeter of the conductive pad. The involuted peripheral edge includes a depth and a width. In one embodiment, the depth of the involuted edge may be in the range of about 30% to about 100% of the width of the involuted edge.

In one embodiment of the present disclosure, the ratio of the perimeter of the conductive pad is a function of the area of the conductive pad.

In one embodiment of the disclosure, the conductive pad is split into at least two sections. In such an embodiment, the conductive pads enable return electrode monitoring circuits to monitor various parameters between the sections of the conductive pad (e.g., temperature, current, contact quality, impedance, etc.). In a related embodiment, the sections of the conductive pad are interlocking.

A method for performing monopolar surgery is also disclosed. The method includes the steps of providing an electrosurgical return electrode, as described above, placing the electrosurgical return electrode in contact with a patient, generating electrosurgical energy via an electrosurgical generator, and supplying the electrosurgical energy to the patient via an active electrode.

An electrosurgical system for performing electrosurgery is also disclosed. The system includes an electrosurgical generator to provide electrosurgical energy, the electrosurgical return electrode, as described above, and an active electrode to supply electrosurgical energy to a patient.

In one embodiment, the electrosurgical system also includes a return electrode monitor (REM). The REM may provide temperature monitoring, current monitoring, impedance monitoring, energy monitoring, power monitoring and/or contact quality monitoring for the electrosurgical return electrode.

### BRIEF DESCRIPTION OF THE DRAWINGS

The above and other aspects and features of the present disclosure will become more apparent in light of the following detailed description when taken in conjunction with the accompanying drawings in which:

FIG. 1 is a schematic illustration of a monopolar electrosurgical system;

FIG. 2 is a plan view of the electrosurgical return electrode of the monopolar electrosurgical system of FIG. 1;

FIG. 3 shows one envisioned shape of an involuted edge of the electrosurgical return electrode of FIG. 2;

FIG. 3A shows another envisioned shape of an involuted edge of the electrosurgical return electrode that is narrower than the involuted edge of FIG. 3;

FIG. 4 is a cross-sectional view of an electrosurgical return electrode coated with a positive temperature coefficient (PTC) material;

FIG. 5 is a cross-sectional view of the electrosurgical return electrode of FIG. 4, which also includes an adhesive layer; and

FIG. 6 is a plan view of an electrosurgical return electrode split into two halves.

### DETAILED DESCRIPTION

Embodiments of the presently disclosed electrosurgical return electrode and method of using the same are described below with reference to the accompanying drawing figures wherein like reference numerals identify similar or identical elements. In the following description, well-known functions or constructions are not described in detail to avoid obscuring the disclosure in unnecessary detail.

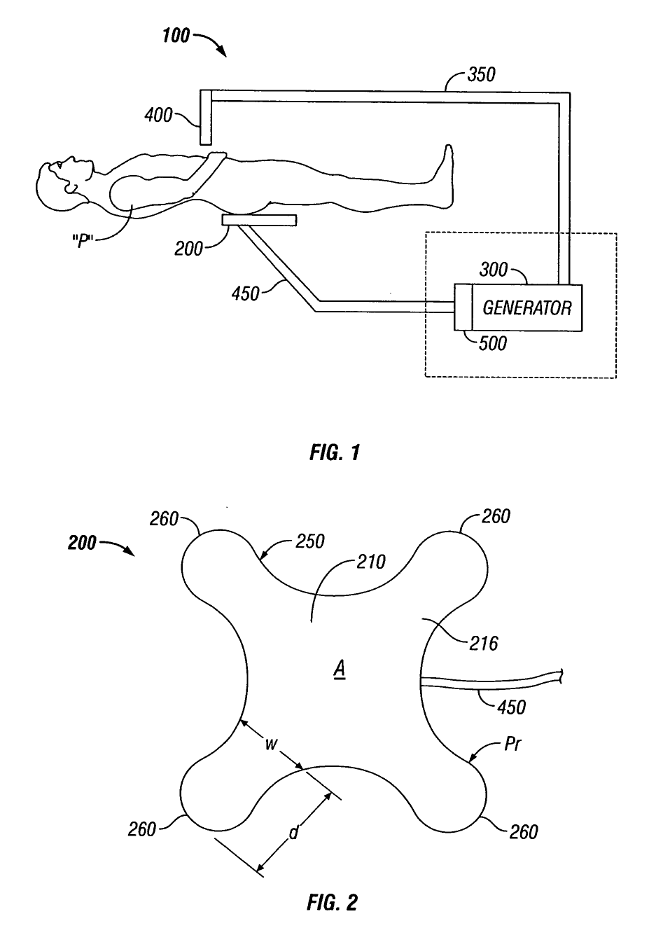

Referring initially to FIG. 1, a schematic illustration of a monopolar electrosurgical system 100 is shown. The electrosurgical system 100 of this embodiment generally includes an electrosurgical return electrode 200, an electrosurgical generator 300, a surgical instrument 400 (e.g., an active electrode) and a return electrode monitor (REM) 500. In FIG. 1, the return electrode 200 is illustrated under a patient “P.” Electrosurgical energy is supplied to the surgical instrument 400 by the generator 300 by a cable 350 to treat tissue (cut, coagulate, blend, etc.). The electrosurgical return electrode 200 acts as a return path for energy delivered by the surgical instrument 400 to the patient “P” and delivers energy back to the electrosurgical generator 300 via a wire 450.

Referring now to FIG. 2, which shows one embodiment of the present disclosure, the electrosurgical return electrode 200 includes a conductive pad 210 having a perimeter “Pr” that defines an area “A” of the conductive pad 210. The portion of the conductive pad 210 that comes into contact with a patient “P” is the patient-contacting surface 216.

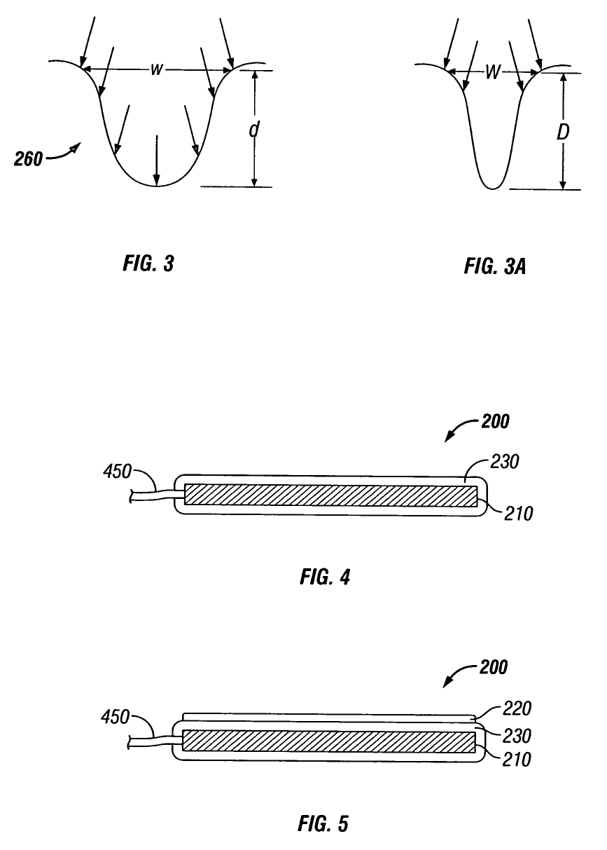

Conductive pad 210 includes an edge 250, having an involuted edge 260 (See FIG. 3). Involuted edge 260 is generally curved and includes a width “w” and a depth “d.” As best shown in FIG. 2, electrosurgical return electrode 200 is illustrated having four (4) involuted edges 260; however, the electrosurgical return electrode 200 may have more or fewer involuted edges 260. Additionally, each involuted edge 260 of the particular pad 200 shown in FIG. 2 has the same width “w” and depth “d.” However, the width “w” and/or depth “d” may vary between each involuted edge 260.

The involuted edges 260 help to distribute current across a longer perimeter of the conductive pad 210, thus mitigating an “edge effect,” where current densities typically increase at the edge of electrosurgical return electrodes 200. Increasing the length of the perimeter “Pr” of the conductive pad 210 by using an involuted edge 260, spreads the current over a larger area and thus reduces the current density of the conductive pad 210 and limits “hot spots.” That is, the use of involuted edges 260, as illustrated in FIGS. 2 and 3 for example, helps to spread the current in a more uniform manner across outer peripheral edges of the conductive pad 210.

More particularly, it has been determined that the shape of each of the involuted edges 260 affects the uniformity of the flow of current. FIGS. 3 and 3A, for example, illustrate different shapes of the involuted edge 260, while the arrows represent the flow of current. In FIG. 3, the current is able to flow to a large portion of the area “A”. By way of contrast, in FIG. 3A, the current is only able to flow to a relatively small portion of the area “A” and does not reach the deepest portion of the involuted edge 260.

It certain embodiments, it may be particularly useful to use involuted edges 260 having a shape similar to that depicted in FIG. 3. Specifically, the size of the depth “d” may be at least 30% of the size of the width “w” and may be in the range of about 30% to about 100% of the size of the width “w.” In a particularly useful embodiment, the depth “d” may be about 50% of the size of the width “w.” The range of sizes for the depth “d” of an involuted edge 260 is from about 0.9 inches to about 1.25 inches. The range of sizes for the width “w” of an involuted edge 260 is from about 1.8 inches to about 2.5 inches. These sizes may vary depending on the particular application of the electrosurgical return electrode 200. The width and depth are depicted in FIG. 3A as “W” and “D,” respectively.

The shape of the involuted edges 260 may also help determine the ratio of the total length of the perimeter “Pr” of the conductive pad 210 to the area “A” of the conductive pad 210. Generally, the involuted edges 260 increase this ratio, as compared to a typical rectangular or circular electrosurgical return electrode.

Now referring to FIG. 4, an electrosurgical return electrode 200 is shown, wherein the conductive pad 210 includes a positive temperature coefficient (PTC) material 230 thereon. The PTC material 230 can be made of, inter alia, a polymer/carbon-based material, a cermet-based material, a polymer material, a ceramic material, a dielectric material, or any combinations thereof. The PTC material 230 acts to distribute the temperature created by the current over the surface of the electrosurgical return electrode 200, which may minimize the risk of a patient burn.

Referring now to FIG. 5, an electrosurgical return electrode 200 is shown, wherein the conductive pad 210 includes a PTC material 230 and an adhesive material 220. The adhesive material 220 is disposed on the patient-contacting surface 216 of the electrosurgical return electrode 200. The adhesive material 220 can be made of, but is not limited to, a polyhesive adhesive, a Z-axis adhesive, a water-insoluble, hydrophilic, pressure-sensitive adhesive, or any combinations thereof. The adhesive material 220 may help to ensure an optimal surface contact area between the electrosurgical return electrode 200 and the patient “P,” which may further limit the possibility of a patient burn. In an embodiment where PTC material 230 is not utilized, the adhesive material 220 may be coupled directly to the electrosurgical return electrode 200.

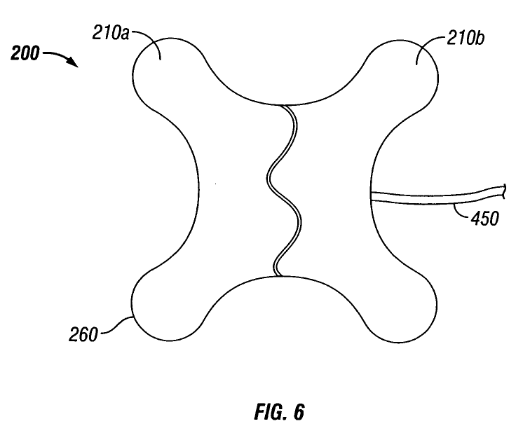

The conductive pad 210 of electrosurgical return electrode 200 may be split into a plurality of sections 210a and 210b, as shown in FIG. 6 with a waved seam. The seam being defined as the gap between sections of electrosurgical return electrode 200. This embodiment enables a return electrode monitor (REM) 500 to monitor various parameters between pad sections 210a and 210b (e.g., temperature, current, contact quality, impedance, etc.). Although not explicitly illustrated, other configurations of electrosurgical return electrode 200 having a plurality of sections are envisioned. For example, electrosurgical return electrode 200 may be split with a seam that is either more or less wavy than the seam illustrated in FIG. 6, including seams that include corners. Additionally, the seam may run in any suitable direction across electrosurgical return electrode 200, including diagonally, vertically, horizontally, etc. Further, the gap between sections of the electrosurgical return electrode 200 may not have a consistent width, i.e., the gap may be wider in some locations and/or narrower in some locations.

The REM circuit 500 has a synchronous detector (not explicitly shown) that supplies an interrogation current sine wave of about 140 kHz across sections 210a, 210b of conductive pad 210 and patient “P”. REM 500 is isolated from the patient “P” via a transformer (not explicitly shown). The impedance in return electrode 200 is reflected back from patient “P” to REM 500 via wire 450. The relationship between temperature and impedance can be linear or non-linear. By measuring the resistance across sections 210a, 210b of conductive pad 210, REM 500 is able to monitor the overall temperature at the return electrode 200 and the contact quality of the return electrode 200. The relationship between temperature and resistance can also be linear or non-linear. In this embodiment, electrosurgical generator 300 would be disabled when the total increase in resistance or temperature of return electrode 200 reaches a predetermined value. Alternatively, there may be several threshold values, such that when a first threshold is exceeded, the output power of electrosurgical generator 300 is reduced, and when a subsequent second threshold value is exceeded, electrosurgical generator 300 is shutdown. This embodiment can be adapted to provide temperature regulation (achievable utilizing a PTC coating), temperature monitoring, current monitoring and contact quality monitoring for return electrode 200, thus greatly reducing the probability of a patient burn.

Wires (illustrated as a single wire 450) return energy from each section 210a and 210b of the conductive pad 210 back to the electrosurgical generator 300. A plurality of wires can be combined to form a single wire 450 (as illustrated in FIG. 6) or can remain as individual wires (not shown) to return energy from the electrosurgical return electrode 200 back to the electrosurgical generator 300.

While several embodiments of the disclosure have been shown in the drawings, it is not intended that the disclosure be limited thereto, as it is intended that the disclosure be as broad in scope as the art will allow and that the specification be read likewise. For example, it is envisioned that the electrosurgical return electrode is substantially symmetrical along both its vertical axis and its horizontal axis. In such an embodiment, rotating the electrosurgical return electrode 90° in either direction will not significantly affect the orientation of the electrosurgical return electrode with respect to the patient. Therefore, the above description should not be construed as limiting, but merely as exemplifications of various embodiments. Those skilled in the art will envision many other possible variations that are within the scope and spirit of the disclosure as defined by the claims appended hereto.

What is claimed is:

1. An electrosurgical return electrode, comprising:
a conductive pad including a perimeter having at least one involuted edge that is configured to reduce a current density of the conductive pad at the perimeter of the conductive pad, the involuted edge including a depth and a width, the depth being at least about 30% of the width.

2. The electrosurgical return electrode according to claim 1, wherein the depth is in the range of about 30% to about 100% of the width.

3. The electrosurgical return electrode according to claim 1, wherein the depth is approximately 50% of the width.

4. The electrosurgical return electrode according to claim 1, wherein the conductive pad comprises a plurality of involuted edges.

5. The electrosurgical return electrode according to claim 1 wherein the conductive pad comprises four involuted edges.

6. The electrosurgical return electrode according to claim 5, wherein the depth of at least one of the four involuted edges is in the range of about 30% to about 100% of the width of the at least one involuted edge.

7. The electrosurgical return electrode according to claim 1, wherein the conductive pad includes a patient-contacting surface and an adhesive material disposed on the patient-contacting surface.

8. The electrosurgical return electrode according to claim 1, wherein the conductive pad is at least partially coated with a positive temperature coefficient (PTC) material.

9. The electrosurgical return electrode according to claim 1, wherein the electrosurgical return electrode is comprised of a plurality of conductive pads.

10. A method for performing monopolar surgery, comprising:
providing an electrosurgical return electrode including a conductive pad including a perimeter having at least one involuted edge that is configured to reduce a current density of the conductive pad at the perimeter of the conductive pad, the involuted edge including a depth and a width, the depth being at least about 30% of the width;
placing the electrosurgical return electrode in contact with a patient;
generating electrosurgical energy via an electrosurgical generator; and
supplying the electrosurgical energy to the patient via an active electrode.

11. The method for performing monopolar surgery according to claim 10, wherein the depth is in the range of about 30% to about 100% of the width.

12. An electrosurgical system for performing electrosurgery on a patient, the electrosurgical system comprising:
an electrosurgical generator to provide electrosurgical energy;
an electrosurgical return electrode including a conductive pad including a perimeter having at least one involuted edge that is configured to reduce current density of the conductive pad at the perimeter of the conductive pad, the involuted edge including a depth and a width, the depth being in the range of about 30% to about 100% of the width; and
an active electrode to supply electrosurgical energy to a patient.

13. The electrosurgical system according to claim 12, wherein the depth o is in the range of about 30% to about 100% of the width.

14. The electrosurgical system according to claim 12, wherein the electrosurgical return electrode is comprised of a plurality of conductive pads.

15. The electrosurgical system according to claim 14, further comprising a return electrode monitor (REM) that monitors at least one of temperature, current, impedance, energy, power and contact quality of the electrosurgical return pad.
